(19) United States
(12) Patent Application Publication
(10) Pub. No.: US 2014/0271345 A1
(43) Pub. Date: Sep. 18, 2014

(54) Sterilization Indicator Tapes, Labels and other Apparatus Made Free of Cellulose, Latex, Lead and Heavy Metals

(71) Applicants: Michael Pavesi, New Boston, NH (US); Daniel Swiger, Sewell, NJ (US)

(72) Inventors: Michael Pavesi, New Boston, NH (US); Daniel Swiger, Sewell, NJ (US)

(21) Appl. No.: 13/999,651

(22) Filed: Mar. 14, 2014

Related U.S. Application Data
(60) Provisional application No. 61/852,345, filed on Mar. 15, 2013.

(51) Int. Cl. A61L 2/28

(52) U.S. Cl. CPC A61L 2/28
USPC 422/1; 436/1

(57) ABSTRACT

A sterilization indicator apparatus is provided. The sterilization indicator apparatus includes a base member having a top side and a bottom side. In a preferred embodiment, the base member is constructed of a plastic material such as a polypropylene. An adhesive is applied to the bottom side of the base member. In one embodiment, a latex-free resin is employed, but other types of adhesives may also be employed. One or more indicator elements are located on the surface opposite the adhesive. The indicator elements may take any shape, such as bars, squares, circles, ellipses, or any desired shape. The indicator elements function as sterilization indicators that are capable of indicating whether or not they have been exposed to a sterilization process.

This application claims priority from U.S. provisional application Ser. No. 61/852,345, filed Mar. 15, 2013, entitled “Sterilization Indicator Tapes, Labels and Other Apparatus Made Free of Cellulose, Latex, Lead and Heavy Metals,” which is incorporated herein by reference in its entirety.

### FIELD OF THE INVENTION

The present invention generally relates to medical devices. More particularly, the invention concerns methods and apparatus to provide sterilization indicator tapes and labels free of cellulose, latex, lead and heavy metals.

### BACKGROUND OF THE INVENTION

In the surgical arena, there are many reusable products that are being used in hospitals and surgery centers that are reprocessed for reuse. These items have been purchased as reusable items from manufacturers who have provided cleaning and sterilization instructions to the facilities. Since many of these devices will be used over and over again as separate items or grouped together in packs, the institution should be able to deliver them sterile to the surgeons and staff doing the surgical procedure. The items processed could range from drapes, towels, sheets, and gowns which are the soft goods required for the surgical case. In addition, many stainless steel, titanium and other metal and composite devices are placed in trays or rigid containers and wrapped which provides a barrier against contamination.

These devices are not disposable devices or listed as “Single Use” and are employed by the institution more than once. It is necessary that reusable instruments be cleaned and otherwise prepared for an additional use. Prior to that happening it is incumbent that the processing arm of the facility render these components sterile.

Due to the number of devices that could or would potentially be involved in this process, it is possible that these items may be sterilized or autoclaved and stored for later use when needed. In the past and in some facilities today sterile processing departments use linen or reusable cloth wrappers. In a transition to disposable wraps, the change in material went to the use of a breathable barrier made of synthetic materials. Whichever material is used, there is a need to identify those items stored as being sterilized. It is imperative that these sterilized packages are marked, tracked and identified to avoid complications in the healthcare setting.

To separate and identify unsterilized products from being used, there are various indicators that are being employed as labels and tape on the outside and internal areas of the packs. For example, sterilization indicators are placed in the wrapper package or attached to the wrapper. The best or most convenient way to indicate and provide closure is to locate the indicator tape on the outside of the package. For example, a pressure sensitive adhesive indication tape is used for holding the cloth or synthetic wrappers in a closed position prior to, during and after sterilization of the processed enclosed product.

Currently, approximately 90% of all wrap used in the healthcare environment is a material based of spun polypropylene. Although laundering cloth or linen wrappers usually in aqueous alkali solutions still may be present in a small number of facilities today, the removal of the tape is indicated to protect the material from being damaged.

Indicator tapes today are based on a paper pressure sensitive adhesive tape, although they were originally based with a printed lead based image. Heavy metals such as copper and other heavy metals are also being used to provide the indication/validation of sterilization.

The “Green” environment that surrounds us in 2012 and beyond, requires another level of indicators for today's healthcare. Therefore, there remains a need to overcome one or more of the limitations in the above-described, existing art.

The discussion of the background to the invention included herein is included to explain the context of the invention. This is not to be taken as an admission that any of the material referred to was published, known or part of the common general knowledge as at the priority date of the claims.

### BRIEF DESCRIPTION OF THE DRAWINGS

FIG. 1 is an exploded perspective view of one embodiment of an indicator tape that includes an adhesive and several indicator bars, the indicator tape embodying the principals of the invention; and

FIG. 2 is an exploded perspective view of the indicator tape illustrated in FIG. 1, with the indicator bars visibly changed.

It will be recognized that some or all of the Figures are schematic representations for purposes of illustration and do not necessarily depict the actual relative sizes or locations of the elements shown. The Figures are provided for the purpose of illustrating one or more embodiments of the invention with the explicit understanding that they will not be used to limit the scope or the meaning of the claims.

### DETAILED DESCRIPTION OF THE PREFERRED EMBODIMENTS

In the following description, for the purposes of explanation, numerous specific details are set forth in order to provide a thorough understanding of the tapes and labels that embody principals of the present invention. It will be apparent, however, to one skilled in the art that the tapes and labels may be manufactured without some of these specific details. Throughout this description, the embodiments and examples shown should be considered as exemplars, rather than as limitations on the tapes and labels. That is, the following description provides examples, and the accompanying drawings show various examples for the purposes of illustration. However, these examples should not be construed in a limiting sense as they are merely intended to provide examples of the tapes and labels rather than to provide an exhaustive list of all possible implementations of the tapes and labels, and the methods of making same.

Specific embodiments of the invention will now be further described by the following, non-limiting examples which will serve to illustrate various features. The examples are intended merely to facilitate an understanding of ways in which the invention may be practiced and to further enable those of skill in the art to practice the invention. Accordingly, the examples should not be construed as limiting the scope of the invention. In addition, reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner in one or more embodiments.

The present invention relates to sterilization tapes and labels utilizing specialized imprinting (heavy metal & latex free) on synthetic materials with pressure sensitive adhesion properties to secure, close, indicate, track or identify completed reprocessing cycles. This tape will be used to secure plastic sheeting, poly-spun fabric as a sterilization barrier to render and maintain a product sterile. This tape will be utilized in the process not only to secure and indicate the complete sterilization cycle of products using thermal, steam, and chemical methods, but also as a part or an additive to enhance the reclamation process.

The Green Initiatives in most hospitals today not only overlook that the tapes now come latex free, but the fact that many more facilities use a lead-based tape that weighs even heavier on the waste stream and the health of our environment. Any type of post reclamation of the products with labels containing heavy metals is extremely difficult, and therefore they never enter the production cycle again.

Couple that with the fact that only 16% of all synthetic sterilization wrap, known in the Operating Room as “Blue Wrap” is even being considered as being eligible for recycling. The Blue Wrap is promoted by the original equipment manufacturer that supplies approximately 90% of the domestic and international markets that the products end of life is a number 5 recyclable membrane.

However, to date, no one has been able to find the solution to the re-use of a number 5 reclaimable plastic sterilization wrap that is filling our landfills, being incinerated because the indicator tape renders it unusable as an recyclable and reclaimable by-product.

The present invention uses a synthetic material comprised of polypropylene or a combination of synthetic materials to be the base material of the tape and labels. Pressure sensitive adhesives are applied to the under section of the tape and labels which need to retain its properties after, or during exposure to moisture and heat that is likely to be encountered during the sterilization process.

The imprinting of the top side of the tape follows a specific process including a specific iconic image printed without heavy metal (lead/copper) that indicates the package is sterile. The tape and labels also enjoy a latex free claim to further promote the efficacy of this product.

In one embodiment, the indicator tapes will be used to both close the sterilization wrapper and indicate and confirm that the articles are sterilized and compliant with all healthcare and FDA regulations (510K submissions/AAMI).

The invention provides the catalyst for a heavy metal free/latex free environment. The tape and labels is now a major contributor to promoting “Green Health” for any institution utilizing the tape to support pre- and post-operative waste management.

In preferred embodiments, this invention provides a method making an indicator tape comprising: (a) polypropylene or similarly compatible base for the tape and labels; (b) heavy metal free ink; (c) latex free resin/pressure sensitive adhesive; (d) an iconic design imprint comprising an indicator ink in combination with a binder on one of the sides in conjunction with a unique application, with the indicator ink being capable of undergoing change when exposed to sterilization conditions; and, (e) coating a pressure sensitive and moisture resistant adhesive on the opposing side of the backing.

In addition, other embodiments of the invention will comprise synthetic based tape and or labels that will be used as a catalyst for rendering other plastics into a reclaimable byproduct that is free of cellulose, latex and lead. The tape/labels will be a compliment to the material that will be shredded, extruded and pelletized for post consumer use. This is important in the re-use of the plastics recovered from the medical industry utilizing plastic barriers. Since this material is of equal composite material to those barrier drapes and wraps that it will be used to secure and indicate the completed sterilization process, it renders the recycled material as “the same.” This enhances the process of being a true unadulterated material to be recycled and natural in the progression of multiple instances of recycling.

The present invention delineates the reclaimed product into a material that can be reused cradle to cradle, thereby reducing or eliminating the product from ever reaching a landfill or incinerator. The present invention provides the byproduct's innate ability to be used over and over again. This process also excludes contamination from other sources, such as, but not limited to, Hevea Latex, lead and other materials that would or could potentially pollute any product that was to be used again in a medical grade material.

The indicator material in the tapes and labels are made in a rolled synthetic tape of various widths and thicknesses. These rolls are coated with pressure sensitive adhesives providing a bond between the actual tape and the barrier wrap that is used to cover devices being sterilized with heat, moisture, and other chemical or mechanical means. The indicator tapes are useful to maintain a sterilization wrapper containing articles (not limited to instruments, power equipment and devices) in a closed position during sterilization during pre- and post-sterilization handling, while proving a means to indicate if the wrapped articles have passed through a sterilization cycle. After use, the chemical imprint will change to a discernible color or shade difference to indicate the completion of the cycle.

In addition, the same methods and processes would apply to the same chemical imprints, testifying to the completion of sterilization cycles for use of synthetic labels of various shapes, thicknesses and sizes. These too will be used as an indicator and can be placed on individual or grouped products needing sterility cycle validation.

The standards for making indicator tapes and labels using cellulose has already been established within the healthcare, pharmaceutical, dental and veterinarian markets. The same methodology of production and application of pressure sensitive adhesive has already been demonstrated as well.

The formation of the prescribed material as in the case of cellulose based tapes is that the sheets of material will be formulated, the pressure sensitive adhesive will be added, and then ink is applied. The tape material and core will be slit to the appropriate width, usually between ¾ of an inch to one (1) inch and to the approximate length of 60 yards. The scope and dimensions of these materials can be of various sizes.

The difference in the process of making this tape is the application of inks and adhesive materials to a plastic based product. The ability of the tape of the present invention in utilizing high performance inks is important in their versatility and their adaptability to be applied to the plastic membrane.

Labels will also go through the same process as the tape, after which they will be produced in different geometric configurations and they will be printed in the same format as other labels.

As discussed, the sterilization indicator tape and labels of the present invention are made from a synthetic plastic base material, and have a pressure sensitive adhesive coating that utilizes inks, solvents and other additives in different viscosities to identify the completion of a sterilization cycle. These sterilization methods are exposed to the following thermal, water and chemical based processes to change the coloration of the inks upon cycle completion.

The indicator tape may be made to the current specifications of: width ¾″-1″ to a overall length of 60 yards (55 meters). The thickness of the tape will vary but will normally range from 3 millimeters to 7 millimeters in thickness. In addition the tape thickness will incorporate the adhesive layer, preferably 1 millimeter thick as well as an ink application. This tape will be constructed from a material base that is, in part, or in whole, of a poly-propylene or other #5 coded plastic. This plastic material will have similar melting and extruding properties.

The indicator tape itself will be free from all latex (hevea based) as provided in the ASTM standards regulating latex protein levels. The Lowrey method will be used to ensure that the tape, adhesive and inks conform to applicable standards.

The material used for the indicator tape will be of petroleum based virgin or recycled plastic and can contain varied amounts of #5 plastic. These plastic compounds will either be combined in various amounts, blended for performance characteristics or will be pure with regard to virgin or recycled material. This will allow for the process of shredding, extruding or pelletizing of the material from an originator (new product) or a recyclable product (end of life). The material can be pure in content or combinations of virgin and recyclable or reclaimable materials.

The indicator tape can be made from biodegradable material made up from plant life, synthetics and commercially developed material that will biodegrade within the environment.

As illustrated in FIG. 1, the steam indication tape works by subjecting the wrapped package through a specific process that exposes the package to heat and moisture over time. The tape acts not only as the mechanism that binds the package together, but provides a physical change in coloration to indicate sterility. These standards are set by several agencies but the most prestigious is in accordance with AAMI standards.

Referring now to FIG. 2, the figure shows the ink being convincingly filled in which indicated that the product had served the appropriate time, moisture content and high temperature to render the contents within the package sterile.

The high performance ink materials are used to produce a visual change in the characteristic of the inks from a true color change (i.e. blue to soft brown) where a designated change occurs at the completion of the sterilization cycle. In addition, the indication of a complete sterilization cycle may also be indicated if the colorization changes in shade intensity. This example would be from a red to a softer orange/red, or black to grey to prove cycle validity. The range of colors will be based on a combination of the three primary colors and combinations thereof to distinguish between unexposed and exposed inks/material. This is disclosed in the following Table 1:

TABLE 1
Example Sterilization Chart of colors for specific processes

| Dry Heat | | Ethylene Oxide | |
|---|---|---|---|
| Blue to Brown | Purple to Brown | Blue to Brown | Dark Blue to |
| | | | Soft Brown |
| Blue to Green | Green to Soft Green | White to brown | |
| Green to Brown | Pink to Brown | Red to Yellow | |

| Steam | | Gamma Irradiation |
|---|---|---|
| White to Black | Blue to Black | Yellow to Red |
| Yellow to Black | Gray to Orange | |
| Blue to Strawberry | | |

The inks used in the sterilization process utilizing should be able to endure the following: heat or thermal conditions; gases such as ethylene oxide; and steam utilizing water and thermal or heat conditions to render the wrapped articles sterile. The inks should be reliable in quality and consistent in color and intensity. The inks should be versatile to meet the adherence to different plastic and biodegradable membranes.

The high performance inks will be of water based and of solvent based materials. The tape itself being of plastic or biodegradable material should be able to accept the ink and bond to the tape material. The ink should be suitable to print on various tape materials such as but not limited to polyester, poly-propylene and poly-ethylene, paper and Tyvek tapes. These inks should meet or exceed EN ISO 11140-1 standards for indicator tapes and labels.

The tape and labels that will be used in the present invention will be lead free. The removal of lead from the environment and future products rendered by the reclamation of this tape for the process of reuse in any other format will be 100%. This will minimize or eliminate heavy metals from the waste stream. It is also imperative that any residual lead is not found in the re-use of the reclaimed recycled material in medical grade products. Further, it is important to note that the materials used to build commercial parts, products that come into contact with individuals such as but not limited to toys, handles, drinking vesicles will be lead free.

The tape and label bodies will be of varied colors to not only identify products by color codes but also as a distinguishing color from cellulose based to synthetic based materials. These will be utilized in the same manner that gloves can be distinguished between latex and latex free gloves so that those medical personnel will be able to readily distinguish it from other base materials.

Collection process flow will be influential on the cradle to grave aspects of recycling the indicator tapes and labels. In itself, the process of reclamation starts at the initial point of application. This position applies the indicator tape or label onto an article or articles to be sterilized. Once the tape or label is applied with a pressure sensitive backing for holding properties it is with this that we look for the process to be underway. Storage of said articles is typical. In the removal of this sterilization wrap from the articles it is appropriate to maintain the wrap materials and the tape/labels in a non-contaminated manner.

This process would be demonstrated by following sterile protocol in those situations that demanded that action. In the process of unwrapping sterile trays and sets the material would be handled through a process and procedure that would be conducive to the environment that it was first collected in. That said, other plastics may be handled in a health care facility utilizing proper Personnel Protection Equipment (PPE's) that protect the collector from contamination or causing further contamination to other sources.

Collection in the facility should be segregated from all other infectious waste and processed in the regular waste management flow until it reaches the final collection container. The devices can be carried off the property in a single waste stream process where upon landing at the sorting site. Removal from this sorting process, followed up by the segregation of the material for reclamation will be followed by procedure. It will be provided in writing along with a clinical pathway audit that will identify appropriate waste management practices for all waste streams including but not limited to Pharma-waste, Regulated Medical Waste and Solid Medical waste to any and all facilities in the implementation. The recyclable nature of all waste streams will be reduced to its lowest common denominator.

Referring again to FIGS. 1 and 2, in one embodiment, a sterilization indicator apparatus 10 is illustrated. The sterilization indicator apparatus 10 comprises a base member, or primary tape or label body 15 having a top side and a bottom side. In a preferred embodiment, the base member 15 is constructed of a plastic material such as a polypropylene. However in other embodiments, a biodegradeable plastic, a polymer, a polyester, a polyolefin, a polycarbonate, a polyamide, a polyether, a polyethylene, a polytetrafluoroethylene, a polyurethane, a polyvinyl chloride, a polystyrene, and a combination of two or more thereof may also be employed.

An adhesive 20 is applied to the bottom side of the base member 15, with the adhesive 20 preferably comprising a pressure-sensitive type of adhesive, but other types may also be employed. In one embodiment, a latex-free resin is employed, but other types of adhesives may also be employed.

As showing FIGS. 1 and 2, one or more indicator elements or bars 25 are located on the surface opposite the adhesive 20. The indicator elements 25 may take any shape, such as bars, squares, circles, ellipses, or any desired shape. The indicator elements 25 function as sterilization indicators that are capable of indicating whether or not they have been exposed to a sterilization process. For example, sterilization processes may be a heating process, a steaming process, a chemical process, and a combination of two or more thereof.

Preferably, the indicator elements 25 are manufactured of a heavy-metal-free ink as described above. Referring now to FIG. 2, the indicator elements 25 have changed in appearance and thus indicate they have been exposed to a sterilization procedure. As described above, the change in appearance may employ any type of visible change. For example, TABLE 1 lists several different color changes. However, the indicator elements may only change in a color shade, or may indicate another type of appearance change.

Another embodiment of the present invention comprises a method of sterilizing a plurality of items, the method comprising the steps of providing a sterilization element sized to contain the plurality of items, placing the plurality of items in the sterilization element, sealing the sterilization element with a sterilization indicator tape that includes a sterilization exposure element, exposing the sterilization element to a sterilization process and verifying the sterilization process is complete by determining that the sterilization exposure element has undergone a change in state.

For example, a sterilizable pouch for surgical instruments may have the sterilization indicator apparatus 10 applied onto an upper or other surface of the pouch. Alternatively, the sterilization indicator apparatus 10 in the form of a tape may be employed to seal the sterilization pouch. The pouch is then exposed to a sterilization process and the sterilization process is determined as complete by visually indentifying that the sterilization indicator apparatus 10, and specifically, the indicator elements 25 have undergone a change in state, or appearance.

In a preferred embodiment the sterilization indicator apparatus 10 comprises a roll of tape, the tape comprising a base member 15 having a top side and a bottom side, with the tape manufactured of a plastic material. An adhesive 20 is coupled to the bottom side of the base member 15 and one or more sterilization indicator elements 25 are located on the top side of the base member 15, the sterilization indicator elements 25 capable of indicating whether or not the sterilization indicator apparatus 10 has been exposed to a sterilization process.

Other embodiment sterilization indicator apparatus 10 may comprise a plurality of discrete labels that are manufactured in a roll, and easily removable from the roll.

It is to be noticed that the term “comprising”, used in the claims, should not be interpreted as being limitative to the means listed thereafter. Thus, the scope of the expression “a device comprising means A and B” should not be limited to devices consisting only of components A and B. It means that with respect to the present invention, the only relevant components of the device are A and B.

Similarly, it is to be noticed that the term “coupled”, also used in the claims, should not be interpreted as being limitative to direct connections only. Thus, the scope of the expression “a device A coupled to a device B” should not be limited to devices or systems wherein an output of device A is directly connected to an input of device B. It means that there exists a path between an output of A and an input of B which may be a path including other devices or means.

The terms “an embodiment”, “embodiment”, “embodiments”, “the embodiment”, “the embodiments”, “one or more embodiments”, “some embodiments”, and “one embodiment” mean “one or more (but not all) embodiments of the present invention(s)” unless expressly specified otherwise.

The terms “including”, “comprising” and variations thereof mean “including but not limited to”, unless expressly specified otherwise.

Thus, it is seen that sterilization indicator tapes and labels, the methods and processes for making same are provided.

One skilled in the art will appreciate that the present invention can be practiced by other than the above-described embodiments, which are presented in this description for purposes of illustration and not of limitation. The specification and drawings are not intended to limit the exclusionary scope of this patent document. It is noted that various equivalents for the particular embodiments discussed in this description may practice the invention as well. That is, while the present invention has been described in conjunction with specific embodiments, it is evident that many alternatives, modifications, permutations and variations will become apparent to those of ordinary skill in the art in light of the foregoing description. Accordingly, it is intended that the present invention embrace all such alternatives, modifications and variations.

What is claimed is:

1. A sterilization indicator apparatus, comprising:
a base member having a top side and a bottom side;
an adhesive coupled to the bottom side of the base member; and
a sterilization indicator located on the top side of the base member, the sterilization indicator capable of indicating whether or not the sterilization indicator apparatus has been exposed to a sterilization process.

2. The sterilization indicator apparatus of claim 1, where the base member is comprised of a plastic material, the plastic material selected from a group consisting of: a polypropylene, a biodegradeable plastic, a polymer, a polyester, a polyolefin, a polycarbonate, a polyamide, a polyether, a polyethylene, a polytetrafluoroethylene, a polyurethane, a polyvinyl chloride, a polystyrene, and a combination of two or more thereof.

3. The sterilization indicator apparatus of claim 1, where the sterilization indicator apparatus comprises a tape or a label.

4. The sterilization indicator apparatus of claim 1, where the sterilization process is selected from a group consisting of: a sterilization heating process, a sterilization steaming process, a sterilization chemical process, and a combination of two or more thereof.

5. The sterilization indicator apparatus of claim 1, where the sterilization indicator located on the top side of the base member is comprised of a heavy metal free ink.

6. The sterilization indicator apparatus of claim 1, where the sterilization indicator located on the top side of the base member is comprised of at least one substantially rectangular shaped element.

7. The sterilization indicator apparatus of claim 1, where the sterilization indicator located on the top side of the base member is comprised of at least one substantially rectangular shaped element that includes an indicator ink capable of undergoing a visible change when exposed to the sterilization process.

8. The sterilization indicator apparatus of claim 1, where the sterilization indicator located on the top side of the base member is comprised of:
at least one substantially rectangular shaped element that includes an indicator ink, the indicator ink capable of undergoing a visible change when exposed to the sterilization process, where the visible change comprises a change in a color of the indicator ink or a change in a color shade of the indicator ink.

9. The sterilization indicator apparatus of claim 1, where the adhesive coupled to the bottom side of the base member is selected from a group consisting of: a latex free resin, a pressure sensitive adhesive, and a combination of both a latex free resin and a pressure sensitive adhesive.

10. A method of sterilizing a plurality of items, the method comprising the steps of:
providing a sterilization element sized to contain the plurality of items;
placing the plurality of items in the sterilization element;
sealing the sterilization element with a sterilization indicator tape that includes a sterilization exposure element;
exposing the sterilization element to a sterilization process; and
verifying the sterilization process is complete by determining that the sterilization exposure element has undergone a change in state.

11. The method of sterilizing of claim 10, where the change in state of the sterilization exposure element comprises a change in color or a change in a color shade.

12. The method of sterilizing of claim 10, where the sterilization exposure element comprises at least one substantially rectangular element comprised of a heavy metal free ink.

13. The method of sterilizing of claim 10, where the sterilization indicator tape is comprised of a plastic material, the plastic material selected from a group consisting of: a polypropylene, a biodegradeable plastic, a polymer, a polyester, a polyolefin, a polycarbonate, a polyamide, a polyether, a polyethylene, a polytetrafluoroethylene, a polyurethane, a polyvinyl chloride, a polystyrene, and a combination of two or more thereof.

14. The method of sterilizing of claim 10, where the sterilization indicator tape includes an adhesive selected from a group consisting of: a latex free resin, a pressure sensitive adhesive, and a combination of both a latex free resin and a pressure sensitive adhesive.

15. A sterilization indicator apparatus, comprising:
a roll of tape, the tape comprising a base member having a top side and a bottom side, the tape manufactured of a plastic material;
an adhesive coupled to the bottom side of the base member; and
a sterilization indicator located on the top side of the base member, the sterilization indicator capable of indicating whether or not the sterilization indicator apparatus has been exposed to a sterilization process.

16. The sterilization indicator apparatus of claim 15, where the plastic material is selected from a group consisting of: a polypropylene, a biodegradeable plastic, a polymer, a polyester, a polyolefin, a polycarbonate, a polyamide, a polyether, a polyethylene, a polytetrafluoroethylene, a polyurethane, a polyvinyl chloride, a polystyrene, and a combination of two or more thereof.

17. The sterilization indicator apparatus of claim 15, where the sterilization process is selected from a group consisting of: a sterilization heating process, a sterilization steaming process, a sterilization chemical process, and a combination of two or more thereof.

18. The sterilization indicator apparatus of claim 15, where the sterilization indicator located on the top side of the base member comprises:
at least one substantially rectangular shaped element that includes a heavy-metal-free ink that is capable of undergoing a visible change when exposed to the sterilization process, where the visible change comprises a change in a color of the heavy-metal-free ink or a change in a color shade of the heavy-metal-free ink.

19. The sterilization indicator apparatus of claim 15, where the adhesive coupled to the bottom side of the base member is selected from a group consisting of: a latex free resin, a pressure sensitive adhesive, and a combination of both a latex free resin and a pressure sensitive adhesive.
